(19) United States
(12) Patent Application Publication
(10) Pub. No.: US 2022/0111227 A1
(43) Pub. Date: Apr. 14, 2022

(54) SYSTEMS AND METHODS FOR DELIVERING LOW-LEVEL ELECTROMAGNETIC RADIATION TO A PATIENT

(71) Applicant: Jonathan A. Shanker, Clayton, MO (US)

(72) Inventor: Jonathan A. Shanker, Clayton, MO (US)

(21) Appl. No.: 17/070,392

(22) Filed: Oct. 14, 2020

(51) Int. Cl. A61N 5/06

(52) U.S. Cl. CPC A61N 5/0613; A61N 2005/0659; A61N 2005/0638; A61N 2005/0634

(57) ABSTRACT

A device for delivering low-level electromagnetic radiation to a patient is provided herein. The device includes a support including a base surface that is substantially planar and configured to rest on a substrate. The support includes a contact surface opposite the base surface and contoured to support a curvature of a lumbar region and a sacral region of the patient in a supine position. The device also includes a first plurality of emitters coupled to the support proximate to the contact surface and configured to emit low-level electromagnetic radiation transdermally to at least one of the lumbar region and the sacral region.

### BACKGROUND

The subject matter described herein relates generally to medical devices and, more particularly, to devices and methods for treatment of lower back pain (e.g., lumbago) by positioning a patient to induce target treatment areas into a receptive state for receiving therapeutic amounts of electromagnetic therapy, and delivering therapeutic amounts of electromagnetic therapy to the treatment areas of the patient so positioned.

At least some known lower back pain is caused by injury to muscles, tendons, and/or ligaments in the lumbar region of the spine. Portions of the lower back muscles, such as the multifidus, iliocostalis lumborum, and longissimus muscles, may become strained when muscle fibers are abnormally stretched or torn. At least some known lower back pain is due to activities that cause undue stress to the lumbar region, such as, for example, heavy lifting and/or sudden movements. Lower back pain may range from mild to moderate to severe pain. Patients with lower back pain frequently exhibit symptoms of pain, stiffness, and limited range of mobility in the lumbar region (e.g., difficulty bending, sitting, walking).

At least some known devices, such as contoured support cushions, seat rests, and back braces are configured to reduce the discomfort of lower back pain by supporting the patient in a fashion that relaxes the associated muscle groups and other tissues. However, the relief afforded by such known devices is often temporary, and the devices are not generally capable of healing the affected muscles and other tissues.

Alternatively, at least some known devices are configured to reduce discomfort by administering low dosages of electromagnetic radiation (e.g., near-infrared light) to an affected region. For example, the low-level electromagnetic radiation (often referred to as “low-level light therapy”) is applied by emitters positioned on a wrap or handheld device. However, such devices are known to produce inconsistent results, both among different patients and across different treatment sessions by a single patient.

Accordingly, a device that reduces the discomfort of lower back pain, promotes healing of the underlying muscles and other tissue, and is capable of producing consistent results both among different patients and across treatment sessions for a given patient would find utility.

### BRIEF SUMMARY

In one aspect, a device for delivering low-level electromagnetic radiation to a patient is provided herein. The device includes a support including a base surface that is substantially planar and configured to rest on a substrate. The support includes a contact surface opposite the base surface and contoured to support a curvature of a lumbar region and a sacral region of the patient in a supine position. The device also includes a first plurality of emitters coupled to the support proximate to the contact surface and configured to emit low-level electromagnetic radiation transdermally to at least one of the lumbar region and the sacral region.

In another aspect, a method of operating a device for delivering low-level electromagnetic radiation is provided herein. The method includes positioning the device underneath a patient lying in a supine position on a substrate. The device includes a support including a base surface that is substantially planar and configured to rest on the substrate. The device also includes a contact surface opposite the base surface and contoured to support a curvature of a lumbar region and a sacral region of the patient in the supine position. The device also includes a first plurality of emitters coupled to the support proximate to the contact surface and configured to emit low-level electromagnetic radiation. The method also includes emitting low-level electromagnetic radiation from the first plurality of emitters transdermally to at least one of the lumbar region and the sacral region.

The features, functions, and advantages described herein may be achieved independently in various embodiments of the present disclosure or may be combined in yet other embodiments, further details of which may be seen with reference to the following description and drawings.

### BRIEF DESCRIPTION OF THE DRAWINGS

FIG. 1 is a schematic perspective view of an exemplary device for delivering low-level electromagnetic radiation to one or more treatment areas of a patient;

FIG. 2 illustrates a patient using the exemplary device shown in FIG. 1;

FIG. 3 is a schematic perspective view of an exemplary alternative embodiment of the device shown in FIG. 1;

FIG. 4 is a schematic perspective view of the exemplary alternative embodiment of the device shown in FIG. 3, showing the device in an expanded configuration; and

FIG. 5 is a schematic perspective view of another exemplary alternative embodiment of the device shown in FIG. 1.

### DETAILED DESCRIPTION

The devices and methods described herein position a patient to induce muscles and other tissues associated with lower back pain into a specifically receptive state for receiving therapeutic low-level amounts of electromagnetic radiation, and also deliver therapeutic amounts of low-level electromagnetic radiation to the treatment areas of the patient so positioned. More specifically, such therapeutic low-level electromagnetic radiation, for example near infrared (“NIR”) light, has been found to more effectively and consistently promote healing of muscle tissue, such as in the context of therapy for sports-related injuries, when the targeted muscle fibers are in a gently stretched state, rather than constricted or completely relaxed. The devices and methods described herein position the patient in a fashion that induces muscle fibers in the lumbar region into this gently stretched yet unforced receptive state, and simultaneously orient emitters to apply low-level light therapy transdermally to the muscles and other tissues that have been induced into the receptive state, thereby not only reliably and consistently alleviating pain associated with the lumbar region, but also facilitating healing of the targeted muscles and other tissues to reduce or eliminate the incidence of tension and pain in the lower back. Without wishing to be limited to a particular theory, low-level light therapy is believed to affect a biological change in tissue by inducing a photochemical reaction in the cell, a process referred to as photobiomodulation. For example, low-level light therapy increases the temperature in the muscles and tissues of a treatment area, and improves blood circulation to the treatment area.

The devices and methods described herein include a support having a contact surface contoured to support the lumbar region (i.e., lumbar curvature of the spine) and the sacral region (i.e., sacral curvature of the spine) of the patient's lower back region when the patient lies in a supine position. More specifically, the support is configured to gently stretch areas of the patient's lower back muscles into the light therapy-receptive state described above. The devices and methods further include emitters coupled to (e.g., embedded in) the support proximate to the contact surface and oriented to emit low-level electromagnetic radiation (e.g., NIR light) transdermally into the targeted muscle groups while they are in the receptive state.

In some embodiments, the support includes first and second support elements that are movable relative to each other to adjust a length of the support in a direction generally parallel to the patient's spine. More specifically, the first and second elements are movable relative to each other to adjust a spacing between a first portion of the contact surface, supporting the lumbar region, and a second portion of the contact surface, supporting the sacral region. Accordingly, the device is easily adjustable to each specific patient's lower back length in order to achieve the desired receptive state of the target muscle groups and/or the desired alignment of the emitters and the target muscle groups.

As used herein, an element or step recited in the singular and preceded with the word “a” or “an” should be understood as not excluding plural said elements or steps, unless such exclusion is explicitly stated. Further, references to an “embodiment” or an “implementation” are not intended to be interpreted as excluding the existence of additional embodiments or implementations that also incorporate the recited features. Moreover, unless explicitly stated to the contrary, embodiments or implementations “comprising,” “including,” or “having” an element or a plurality of elements having a particular property may include additional such elements not having that property.

Unless otherwise indicated, approximating language, such as “generally,” “substantially,” and “about,” as used herein indicates that the term so modified may apply to only an approximate degree, as would be recognized by one of ordinary skill in the art, rather than to an absolute or perfect degree. Accordingly, a value modified by a term or terms such as “about,” “approximately,” and “substantially” is not to be limited to the precise value specified. In at least some instances, the approximating language may correspond to the precision of an instrument for measuring the value. Additionally, unless otherwise indicated, the terms “first,” “second,” etc. are used herein merely as labels, and are not intended to impose ordinal, positional, or hierarchical requirements on the items to which these terms refer. Moreover, reference to, for example, a “second” item does not require or preclude the existence of, for example, a “first” or lower-numbered item or a “third” or higher-numbered item.

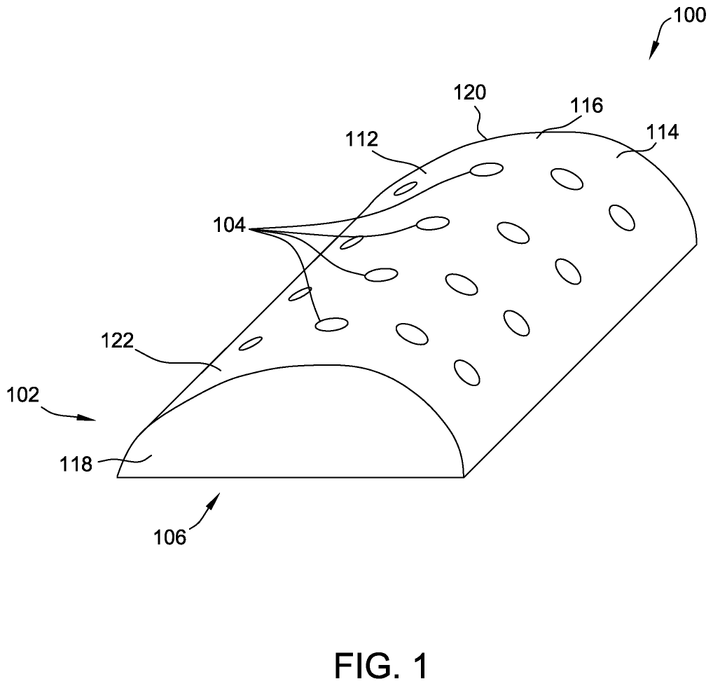

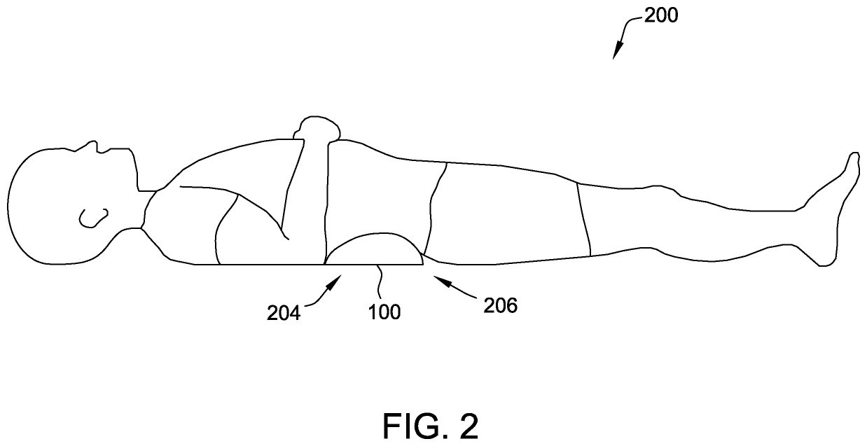

FIG. 1 is a schematic perspective view of an exemplary device 100 for delivering low-level electromagnetic radiation to one or more treatment areas of a patient. FIG. 2 illustrates an exemplary patient 200 using device 100. Device 100 includes a support 102 and a plurality of infrared emitters 104 coupled to support 102. Support 102 includes a base surface 106 and a contact surface 116. With reference to FIGS. 1 and 2, base surface 106 is substantially planar and configured to rest on a substrate (e.g., a floor). Contact surface 116 is opposite base surface 106 and contoured to support a curvature of a lumbar region 204 and a curvature of sacral region 206 of patient 200 in a supine position. Infrared emitters 104 are coupled to support 102 proximate to contact surface 116. Infrared emitters 104 are configured to emit low-level electromagnetic radiation transdermally to at least one of lumbar region 204 and sacral region 206.

In the exemplary embodiment, a first portion of contact surface 116, designated first contact surface 112, is contoured to support the curvature of lumbar region 204. More specifically, in the exemplary embodiment, first contact surface 112 is a convex surface configured to support the curvature of the lower back region of patient 200. A second portion of contact surface 116, designated second contact surface 114, is configured to support sacral region 206 and, in the exemplary embodiment, to prevent excessive pressure on the bottom of the spine. Second contact surface 114 may be positioned to support the coccyx of patient 200. Alternatively, first contact surface 112 and second contact surface 114 each have any suitable shape that enables device 100 to function as described herein. In the exemplary embodiment, first contact surface 112 tangentially meets second contact surface 114 to support the curvature of the patient's lumbar curvature and sacrum curvature.

In the exemplary embodiment, first contact surface 112 and second contact surface 114 are unitarily formed as a single continuous contact surface 116. Alternatively, first contact surface 112 and second contact surface 114 are formed separately and/or contact surface 116 is other than a single, continuous surface. In various embodiments, contact surface 116 is formed as a separable cover overlying a body of support 102, emitters 104 are embedded in the body directly beneath contact surface 116, and contact surface 116 is configured to be substantially transparent to the low-level electromagnetic radiation emitted by emitters 104. In one example, contact surface 116 is a sheet-like protective film or layer that protects emitters 104 from the environment. Correspondingly, the body of support 102 may be formed from a suitable material that enables device 100 to conform to the patient's lumbar curvature and sacrum curvature when in use, and to return to its original shape when device 100 is not being used, such as a suitable foam or cushion material that enables device 100 to compress, stretch, and conform to the patient's lumbar curvature and sacrum curvature, such that (i) the patient's lower back is supported by device 100 and (ii) infrared emitters 104 are directly adjacent to the target treatment areas of the patient's lower back while device 100 is in use. In alternative embodiments, a suitable form or cushion material used to form support 102 may also define contact surface 116. The suitable material may be, for example, a synthetic foam, gel, or fluid material that enables device 100 to function as described herein.

As shown in FIG. 1, base surface 106 is a substantially elongate planar surface configured to rest on a substrate surface (for example, a floor, treatment table, or bed) when device 100 is utilized by the patient. Device 100 includes side surfaces 118, 120. First side surface 118 is transversally oriented to base surface 106 and extends therefrom. Second side surface 120 is parallel to, and opposite from, first side surface 118. Second side surface 120 is transversally oriented to base surface 106 and extends therefrom. Alternatively, base surface 106 and side surfaces 118, 120 each have any suitable shape and/or orientation that enables device 100 to function as described herein.

Emitters 104 are coupled to support 102 proximate to contact surface 116 and oriented to emit energy transdermally to patient 200. In the exemplary embodiment, emitters 104 are embedded in contact surface 116, such that a patient-facing surface of each emitter 104 is flush with contact surface 116. Alternatively, emitters 104 are coupled to support 102 in any suitable fashion that enables device 100 to function as described herein. In the illustrated embodiment, emitters 104 are positioned across contact surface 116 in an arrangement that targets (i.e., that positions specific emitters 104 directly adjacent to) specific muscles in lumbar region 204 and sacral region 206, such as, for example, but not limited to, positions of the multifidus, iliocostalis lumborum, and longissimus muscles, when patient 200 is positioned on device 100. Device 100 may include any suitable number of emitters 104 positioned in any suitable arrangement across first contact surface 112 and second contact surface 114 for effectively administering light to the target treatment areas.

With reference to FIGS. 1 and 2, when a patient lies in a supine position with device 100 positioned underneath the patient's lower back region, device 100 is configured to apply a gentle stretch to the patient's lower back such that target treatment areas (e.g., one or more positions of the multifidus, iliocostalis lumborum, and longissimus muscles) align with one or more infrared emitters 104 of device 100 without requiring the patient to adjust placement of device 100. In the exemplary embodiment, device 100 is configured to slightly extend (e.g., stretch) the patient's lower back when the patient uses device 100, such that the muscles are in a state of increased receptiveness to benefit from light therapy treatment. In the exemplary embodiment, emitters 104 align with target muscles in lumbar region 204 and sacral region 206, in the light therapy-receptive state induced by support 102, to safely and effectively deliver near-infrared light. For example, when emitters 104 are positioned directly adjacent to the target muscles, emitters 104 increase the temperature of the muscles over a period of time, thereby promoting blood circulation to these muscles, activating metabolism, and relieving muscle tension.

In the exemplary embodiment, a first subset of emitters 104 are coupled to support 102 proximate to first contact surface 112 and are configured to emit low-level electromagnetic radiation transdermally to lumbar region 204, and a second subset of emitters 104 are coupled to support 102 proximate to second contact surface 114 and are configured to emit low-level electromagnetic radiation transdermally to sacral region 206. In various embodiments, each of the first and second subsets of emitters 104 is independently activatable. Alternatively, emitters 104 are grouped and/or independently activatable in any suitable arrangement of subsets that enables device 100 to function as described herein

In the exemplary embodiment, emitters 104 are operable to emit low levels of near infrared (“NIR”) light. Emitters 104 may be light emitting diodes (“LEDs”), such as, for example, near-infrared LEDs. Emitters 104 may emit light continuously at a given wavelength over a predefined period of time, or alternatively may emit light in pulses at a given wavelength. The frequency, intensity, and/or wavelength of infrared emitters 104 may be fixed or may vary in accordance with signals from a controller (not shown) configured to control the operation of device 100. Emitters 104 may be configured to emit near infrared light at a wavelength within a range of about 200 nanometers (nm) to 1000 nm. In some embodiments, emitters 104 may be configured to emit near infrared light within a range of about 600-850 nm. Alternatively, emitters 104 are configured to emit near infrared light at any suitable range of wavelengths that enables device 100 to function as described herein. In some examples, at least some of the advantages described herein are achieved by emitters 104 providing energy at the above-described wavelengths, on average, in a range of about 1 milliwatt (mW) to 1,000 mW per cm². In certain examples, at least some of the advantages described herein are particularly achieved by emitters 104 providing energy at the above-described wavelengths, on average, in a range of about 5 milliwatt (mW) to 200 mW per cm². In other examples, emitters 104 are configured to provide energy at any suitable wavelength and/or energy level that enables device 100 to achieve at least some of the advantages described herein.

The controller may be in communication with emitters 104, a power source (not shown), and/or a timing mechanism (not shown) to control the frequency, wavelength, intensity, and/or duration of the light emitted by device 100. In certain embodiments, device 100 may include one or more pressure sensors (not shown) associated with first contact surface 112 and/or second contact surface 114 that enable the controller to determine which emitters 104 to automatically turn on based on the pressure detected when device 100 is in contact with the patient's lower back. Additionally or alternatively, device 100 may be configured to vibrate or pulsate to gently apply pressure and massage the patient's treatment areas while light is being administered to these treatment areas.

In alternative embodiments, device 100 may include an external controller (not shown) or a user computing device (not shown), such as a mobile device (e.g., a smart phone) in communication with the controller. For example, the patient may utilize the user computing device to remotely control the settings associated with device 100. In this example, the patient may remotely adjust the duration of a light therapy session (e.g., instruct the controller to automatically turn off after a set time period and/or after a threshold temperature value is exceeded), adjust the intensity of light being delivered, and/or select one or more emitters 104 to emit light for a session. The power source (not shown) may be housed inside device 100, and may be, for example, one or more internal batteries for providing power to emitters 104. However, device 100 may include any suitable power source for providing power, such as, for example, a plug-in power cord.

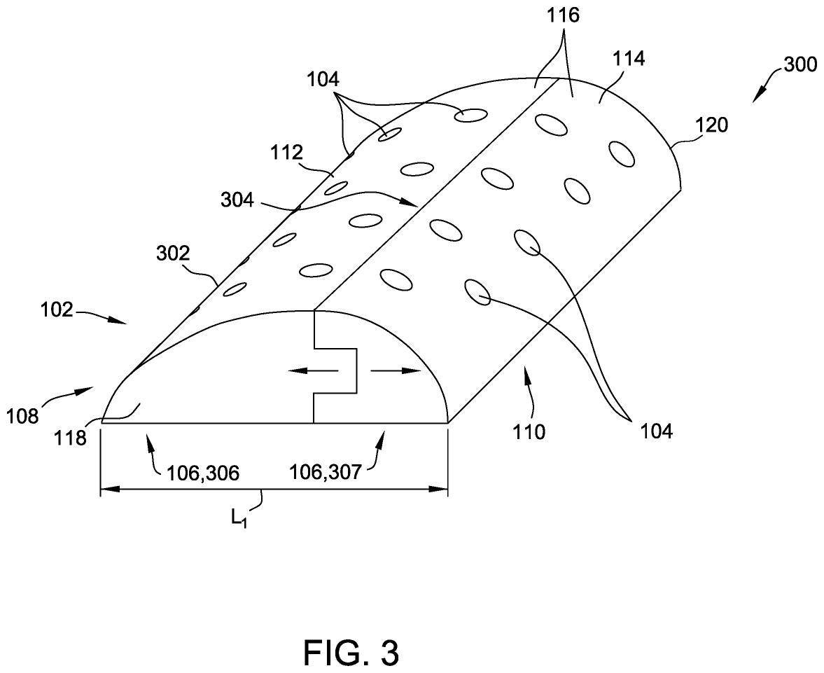

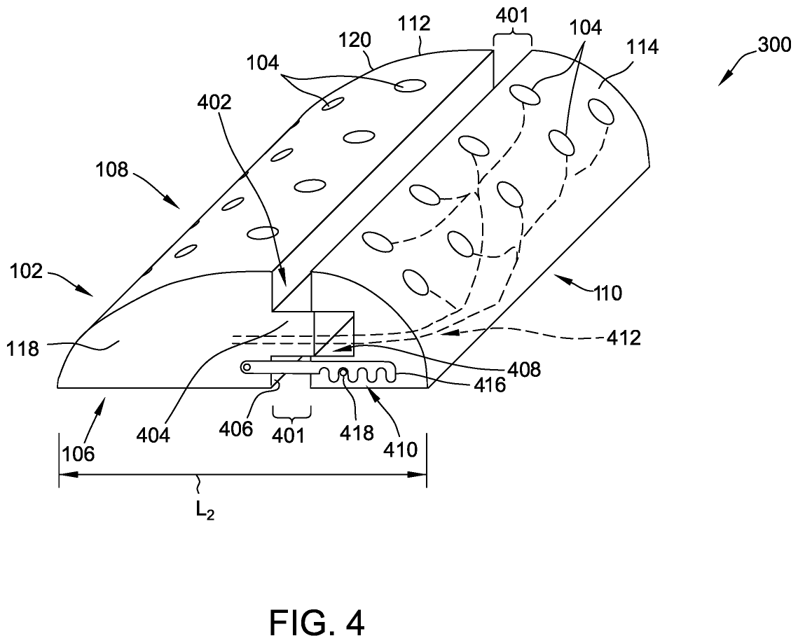

FIG. 3 is a schematic perspective view of an exemplary alternative embodiment of device 100 (shown in FIG. 1), designated device 300. FIG. 4 is a schematic perspective view of device 300 in an expanded configuration. In the exemplary embodiment, support 102 is defined by a first support element 108 and a second support element 110. First support element 108 includes a first portion 306 of base surface 106, and first contact surface 112 is opposite first portion 306 of base surface 106. Second support element 110 includes a second portion 307 of base surface 106, and second contact surface 114, and second contact surface 114 is opposite second portion 307 of base surface 106. First support element 108 and second support element 110 are movable relative to each other to adjust a spacing 401 between first contact surface 112 and second contact surface 114. In other words, device 300 is configured to expand from an original configuration having a first length L₁ to an expanded configuration having a second length L₂ to accommodate patients having different lower back lengths, such as, for example, those with longer than average lumbar spine lengths. In various embodiments, patients may choose to adjust spacing 401 of device 300 to improve simultaneous alignment of emitters 104 on first contact surface 112 with lumbar region 204, and emitters 104 on second contact surface 114 with sacral region 206. For example, a patient with a longer spine may expand gap 401 to simultaneously target various points of the lower back muscles.

In the exemplary embodiment, first support element 108 and second support element 110 are configured to meet at a seam 304 extending from first side surface 118 to second side surface 120. Device 300 is configured to separate at seam 304 and transition from the original configuration to the expanded configuration, such that gap 401 is adjustably defined between first support element 108 and second support element 110. In the exemplary embodiment, seam 304 is generally planar and is generally transverse to side surfaces 118, 120 and to base surface 106. Alternatively, seam 304 has any suitable shape and/or orientation that enables device 300 to function as described herein.

In the exemplary embodiment, support elements 108, 110 are coupled together connected via a connector mechanism 402 that extends across seam 304. In the illustrated embodiment, connector mechanism 402 includes at least one protrusion 404 extending outward from an inner wall 406 of first support element 108 towards second support element 110, and second support element 110 includes at least one recess 408 registered with protrusion 404 and sized to receive protrusion 404 therein in a clearance fit. More specifically, protrusion 404 is configured to slide within recess 408 to maintain a coupling of first support element 108 and second support element 110 when support elements 108, 110 are moved relative to each other. It should be understood that protrusion 404 and recess 408 may be oppositely disposed on second support element 110 and first support element 108, respectively. Alternatively, any suitable connector mechanism 402 may be utilized to movably couple first support element 108 and second support element 110.

Further in the exemplary embodiment, device 300 includes a locking mechanism 410 configured to selectively lock a position of first support element 108 and second support element 110 at a plurality of positions relative to each other. For example, locking mechanism 410 is configured to fix first support element 108 and second support element 110 in place while device 300 is in an expanded configuration, as shown in FIG. 4. In the example embodiment, locking mechanism 410 includes a slotted arm 416 rotatably mounted on first side surface 118 of first support element 108 and a peg 418 fixed on first side surface 118 of second support element 110 and configured to be selectively received by each slot on arm 416. A similar locking mechanism 410 may be provided on second side surface 120 for added stability. Alternatively, locking mechanism 410 may be any suitable mechanism for locking first support element 108 and second support element 110 in position relative to each other, such as, for example, adjustable fasteners, tabs, and geared knobs.

In addition, in the exemplary embodiment, connector mechanism 402 includes a conduit 412 for maintaining control and/or power connections to emitters 104 when first support element 108 and second support element 110 are separated. For example, a power source (e.g., a battery) and/or a controller (not shown) may be housed in first support element 108, and conduit 412 may enable safe and efficient routing of conductive wires for power and/or control signals from first support element 108 to emitters in second support element 110.

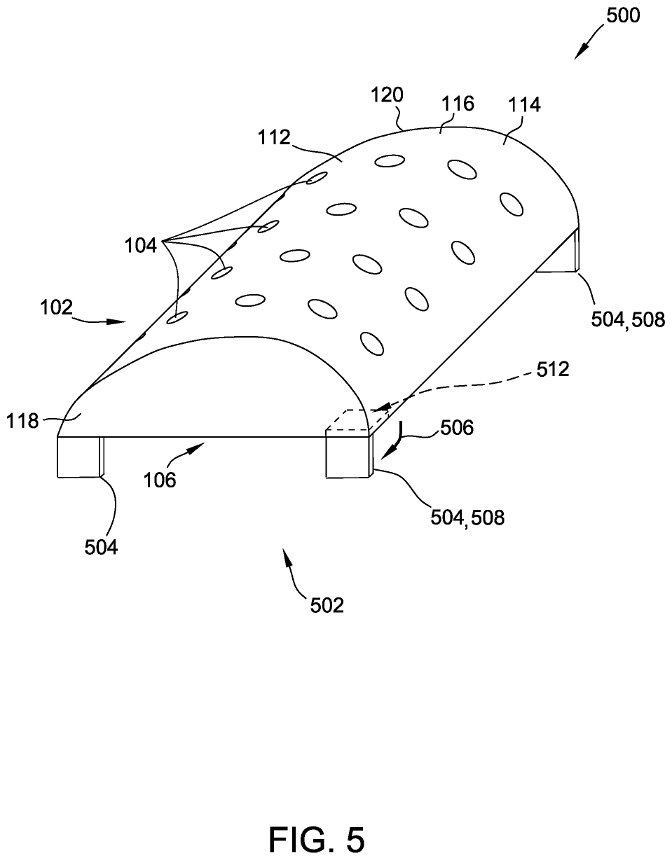

FIG. 5 is a schematic perspective view of another exemplary alternative embodiment of device 100 (shown in FIG. 1), designated device 500. Device 500 is substantially identical to the embodiments of device 100 as described above with respect to FIGS. 1-4, except as described below. In particular, device 500 again includes support 102, which includes base surface 106. In addition, however, device 500 includes an adjustment mechanism 502 operable to vertically adjust portions of contact surface 116 relative to the substrate (e.g., the floor) on which device 500 rests, for example to accommodate physical requirements of a particular patient's lower back in order to achieve the desired receptive state of the target muscle groups and/or the desired alignment of emitters 104 and the target muscle groups.

In the exemplary embodiment, adjustment mechanism 502 includes a plurality of support legs 504 selectively extendable from base surface 106, such that at least a portion of base surface 106 rests indirectly on the substrate. Each support leg 504 may be selectively stowed within support 102 or deployed. For example, each support leg 504 may be rotatable 90 degrees downward, as illustrated by arrow 506, from a complementary stowage pocket 512 defined in base surface 106. Adjustment mechanism 502 is configured to adjust a position of contact surface 116 relative to the floor. In the illustrated embodiment, support legs 504 are provided in all four corners of device 500, enabling elevation of contact surface 116 evenly with respect to the substrate when all four support legs 504 are deployed. In other words, an entirety of contact surface 116 may be raised and lowered relative to the floor to accommodate different physical requirements of different patients. Moreover, solely a pair 508 of support legs 504 may be deployed in order to elevate only a portion of contact surface 116. For example, pair 508 of support legs 504 underneath second contact surface 114 may be deployed, while others of support legs 504 remain stowed in respective stowage pockets 512, such that second contact surface 114 is elevated relative to the floor and relative to first contact surface 112 to accommodate different physical requirements of different patients. Alternatively, 500 includes any suitable adjustment mechanism 502 that enables device 500 to function as described herein.

Although device 300 and device 500 are described and illustrated separately for ease of explanation, it should be understood that in some embodiments, adjustment mechanism 502 is advantageously combined with one or more features of device 300 described above.

Exemplary embodiments of medical devices are described above in detail. The methods and systems are not limited to the specific embodiments described herein, but rather, operations of the methods and components of the systems may be utilized independently and separately from other operations and/or components described herein. For example, the methods and apparatus described herein may have other industrial and/or consumer applications and are not limited to practice with medical devices as described herein. Rather, one or more embodiments may be implemented and utilized in connection with other industries.

This written description uses examples to illustrate the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A device for delivering low-level electromagnetic radiation to a patient, said device comprising:
a support comprising:
a base surface, being substantially planar and configured to rest on a substrate; and
a contact surface, bounded by laterally opposing first and second side surfaces and extending continuously therebetween, said contact surface being opposite said base surface and contoured to support a curvature of a lumbar region and a sacral region of the patient in a supine position; and
a first plurality of emitters coupled to said support proximate to said contact surface at more than two different lateral positions between said first and second side surfaces and configured to emit low-level electromagnetic radiation transdermally to at least one of the lumbar region and the sacral region.

2. The device according to claim 1, wherein said support further comprises:
a first support element comprising a first portion of said base surface and a first portion of said contact surface opposite said first portion of said base surface, said first portion of said contact surface contoured to support the curvature of the lumbar region; and
a second support element comprising a second portion of said base surface and a second portion of said contact surface opposite said second portion of said base surface, said second portion of said contact surface contoured to support the curvature of the sacral region,
wherein said first support element and said second support element are movable relative to each other to adjust a spacing between said first and second portions of said contact surface.

3. The device according to claim 2, wherein said first support element further comprises at least one protrusion extending therefrom towards said second support element, and said second support element further comprises at least one recess registered with said at least one protrusion and sized to receive said at least one protrusion therein in a clearance fit, and wherein said at least one protrusion is configured to slide within said at least one recess to maintain a coupling of said first and second support elements when said first and second support elements are moved relative to each other.

4. The device according to claim 2, further comprising at least one locking mechanism configured to selectively lock a position of said first and second support elements at each of a plurality of positions relative to each other.

5. The device according to claim 1, wherein said first plurality of emitters comprises:
a first subset of emitters configured to emit low-level electromagnetic radiation transdermally to the lumbar region; and
a second subset of emitters configured to emit low-level electromagnetic radiation transdermally to the sacral region.

6. The device according to claim 5, wherein each of said first and second subsets of emitters is independently activatable.

7. The device according to claim 1, wherein:
said support comprises a body and a cover overlaying said body, said cover comprising said contact surface;
said first plurality of emitters is embedded in said body directly adjacent said contact surface; and
said contact surface is substantially transparent to the low-level electromagnetic radiation emitted by said first plurality of emitters.

8. The device according to claim 1, wherein said support is formed from a cushion material that enables said device to compress and conform to the curvature of the lumbar region and the curvature of the sacral region.

9. The device according to claim 1, wherein said first plurality of emitters is configured to emit near infrared light.

10. The device according to claim 1, further comprising an adjustment mechanism operable to vertically adjust portions of said contact surface relative to the substrate.

11. The device according to claim 10, wherein said adjustment mechanism further comprises a plurality of support legs selectively extendable from said base surface, such that at least a portion of said base surface rests indirectly on the substrate.

12. The device according to claim 11, wherein each of said plurality of support legs is stowable in a complementary pocket defined in said base surface.

13. A method of operating a device for delivering low-level electromagnetic radiation, said method comprising:
positioning the device underneath a patient lying in a supine position on a substrate, wherein the device includes:
a support including:
a base surface, being substantially planar and configured to rest on the substrate; and
a contact surface, bounded by laterally opposing first and second side surfaces and extending continuously therebetween, the contact surface being opposite the base surface and contoured to support a curvature of a lumbar region and a sacral region of the patient in the supine position; and
a first plurality of emitters coupled to the support proximate to the contact surface at more than two different lateral positions between the first and second sides and configured to emit low-level electromagnetic radiation; and
emitting low-level electromagnetic radiation from the first plurality of emitters transdermally to at least one of the lumbar region and the sacral region.

14. The method according to claim 13, further comprising adjusting a mechanism of the device to position the device relative to the at least one of the lumbar region and the sacral region of the patient.

15. The method according to claim 13, wherein positioning the device underneath the patient comprises:
positioning a first portion of the contact surface proximate the curvature of the lumbar region; and
positioning a second portion of the contact surface proximate the curvature of the sacral region.

16. The method according to claim 15, wherein the first portion of the contact surface is on a first support element and the second portion of the contact surface is on a second support element, said method further comprising moving the first support element and the second support element relative to each other to adjust a spacing between said first and second portions of the contact surface.

17. The method according to claim 16, wherein the first support element further includes at least one protrusion extending therefrom towards the second support element, and the second support element further includes at least one recess registered with the at least one protrusion and sized to receive the at least one protrusion therein in a clearance fit, and
wherein said method further comprises sliding the at least one protrusion within the at least one recess to maintain a coupling of the first and second support elements when the first and second support elements are moved relative to each other.

18. The method according to claim 17, further comprising adjusting at least one locking mechanism of the device to selectively lock a position of the first and second support elements at each of a plurality of positions relative to each other.

19. The method according to claim 13, wherein the first plurality of emitters includes (i) a first subset of emitters configured to emit low-level electromagnetic radiation transdermally to the lumbar region, and (ii) a second subset of emitters configured to emit low-level electromagnetic radiation transdermally to the sacral region, and
wherein said method further comprises independently activating at least one of the first subset of emitters and the second subset of emitters.

20. The method according to claim 13, further comprising operating an adjustment mechanism to vertically adjust portions of the contact surface relative to the substrate.
